(19) United States
(12) Patent Application Publication
(10) Pub. No.: US 2017/0119744 A1
(43) Pub. Date: May 4, 2017

(54) Pharmaceutical Composition Comprising a Beta-2-Agonist and Anticholinergic Agent

(71) Applicant: Cipla Limited, Mumbai (IN)

(72) Inventors: Geena MALHOTRA, Mumbai (IN); Preeti RAUT, Mumbai (IN)

(73) Assignee: Cipla Limited, Mumbai (IN)

(21) Appl. No.: 15/319,700

(22) PCT Filed: Jun. 18, 2015
(86) PCT No.: PCT/GB2015/000189

§ 371 Date: Dec. 16, 2016

(30) Foreign Application Priority Data
Jun. 18, 2014 (IN) 1969/MUM/2014

(51) Int. Cl. A61K 31/439; A61K 45/06; A61K 9/00; A61K 31/4704

(52) U.S. Cl. CPC A61K 31/439; A61K 31/4704; A61K 45/06; A61K 9/008; A61K 9/0075

(57) ABSTRACT

The present invention relates to pharmaceutical compositions for inhalation which comprise one or more bronchodilators.

### CROSS-REFERENCE TO RELATED APPLICATIONS

This application is a filing under 35 U.S.C. 371 of International Application No. PCT/GB2015/000189 filed Jun. 18, 2015, entitled “Pharmaceutical Composition Comprising a Beta-2-Agonist and Anticholinergic Agent” which claims priority to Indian Patent Application No. 1969/MUM/2014 filed Jun. 18, 2014, which applications are incorporated by reference herein in their entirety.

### FIELD OF INVENTION

The present invention relates to pharmaceutical compositions for inhalation which comprise one or more bronchodilators. There is also provided a process for preparing the pharmaceutical compositions and use thereof in the treatment and/or prevention of respiratory, inflammatory or obstructive airway disease.

### BACKGROUND OF INVENTION

Asthma and chronic obstructive pulmonary disease (COPD) are the common conditions or ailments which affect many people. Airflow obstruction is the main characteristic feature in each of these airway diseases and the medications utilized in the treatment are also often similar.

Asthma is a chronic inflammatory disorder of the airways associated with airway hyper responsiveness, which leads to recurrent episodes of wheezing, breathlessness, chest tightness, and coughing. These episodes are associated with variable airflow obstruction within the lung which is often reversible, either spontaneously or with treatment.

Chronic obstructive pulmonary disease (COPD) is a severe respiratory condition that is increasingly prevalent worldwide. In India, the estimated prevalence is about 12.36 million. It is currently the fourth leading cause of death in the UK & US, and predicted to rank third in the global impact of disease by the year 2020.

Chronic obstructive pulmonary disease (COPD) is a preventable and treatable disease state characterized by air flow limitation that is not fully reversible. The airflow obstruction is usually progressive and associated with an abnormal inflammatory response of the lungs to noxious particles or gases, primarily caused by cigarette smoking. Although COPD affects the lungs it also produces significant systemic consequences. COPD is associated with mucus hyper secretion, emphysema and bronchiolitis.

Therapy for the treatment and/or prevention of asthma and chronic obstructive pulmonary disease (COPD) currently includes the use of bronchodilators such as beta₂-agonists, anticholinergics and steroids.

More specifically asthma, COPD and other related disorders have been known to be treated with beta₂-agonist as they provide a bronchodilator effect, resulting in relief from the symptoms of breathlessness. Beta₂-agonists can be short acting for immediate relief, or long acting for long term prevention of asthma symptoms.

Long acting β₂-agonists improve lung function, reduce symptoms and protect against exercise-induced dyspnea in patients with asthma and COPD. Long acting β₂-agonists induce bronchodilation by causing prolonged relaxation of airway smooth muscle. In addition to prolonged bronchodilation, long acting β₂-agonists (LABAs) exert other effects such as inhibition of airway smooth-muscle cell proliferation and inflammatory mediator release, as well as non-smooth muscle effects, such as stimulation of mucociliary transport, cytoprotection of the respiratory mucosa and attenuation of neutrophil recruitment and activation.

Further, use of a long acting β₂-agonist reduces the frequency of drug administration.

Currently known long acting β₂-agonists (LABAs) include salmeterol, formoterol, indacaterol, olodaterol, carmoterol, milvetrol, abediterol and vilanterol.

Anticholinergic agents or long acting muscarinic antagonists also act as bronchodilators and are potential alternatives to beta agonists. However, anticholinergics can also be administered along with β₂-agonists (LABAs) for the management of asthma. Anticholinergics act by competing with acetylcholine for the receptor sites at vagus nerve or nerve-muscle junctions. This prevents the transmission of reflexes that are induced by asthma stimuli.

Use of anticholinergic drugs is advantageous in elderly patients since, the responsiveness to β₂-agonists declines as age increases. Further, it is also advantageous to use in patients who have developed tolerance with the continuous use of β₂-agonist agents.

Further, anticholinergics can also be used in patients suffering from nocturnal asthma, chronic asthma with concurrent fixed way obstruction, intrinsic asthma and also in patients with asthma of longer duration.

Single drug therapy has always been the treatment of choice for the management of asthma and COPD. LABA's and LAMA's are the standard treatments of choice and they need to be initiated depending on the state of the patient. Further, combination therapy of LABA's and LAMA's is always prescribed for patients who have exacerbations or undergo breathlessness.

The combinations of such LABA's and LAMA's that are currently being developed, such as glycopyrronium and indacaterol, aclidinium and formoterol, aclidinium and glycopyrronium, tiotropium and olodaterol as well as umeclidinium and vilanterol exhibit significant bronchodilation effect.

Further, although it is known that, combination therapy of a β₂-agonist and an anticholinergic agent improves pulmonary efficiency, reduces inflammatory response and provides symptomatic relief as compared to higher doses of inhaled corticosteroid alone in patients affected by respiratory disorders such as asthma, the selection of a specific β₂-agonist and an anticholinergic always plays a very important role in formulation of fixed dose combinations to control respiratory disorders.

Further, the inventors have appreciated that it would be advantageous if the specific β₂-agonist and the anticholinergic agent would demonstrate a sustained duration of action of 24 hours. They have thus investigated various combinations of bronchodilator and β₂-agonist to try and find a combination that provides these advantages.

Additionally, the inventors have appreciated that providing a combination of a bronchodilator and β₂-agonist that demonstrate a sustained duration of action of 24 hours would simplify the therapy and reduce the cost. The inventors have appreciated that reducing the dose frequency to the minimum is a main step in simplifying asthma management for improving patient adherence to the therapy.

WO2012168160 discloses a dry powder inhaler of umeclidinium and also discloses its combination with inhaled corticosteroids as fluticasone propionate, mometasone furoate, ciclesonide, budesonide and fluticasone furoate.

WO2012168161 discloses a combination of umeclidinium and a corticosteroid as fluticasone propionate, mometasone furoate, ciclesonide, budesonide and fluticasone furoate.

WO2013153146 discloses aggregate drug particles of nanoparticulate drug particles of umeclidinium bromide and also discloses the combination of these nanoparticulate drug particles of umeclidinium bromide with vilanterol and fluticasone furoate.

WO2011067212 discloses a combination of umeclidinium and vilanterol as well as umeclidinium, vilanterol and fluticasone furoate.

WO2013178742 discloses dry powder inhaler of abediterol and also discloses its combination with tiotropium, aclidinium, mometasone, fluticasone and roflumilast.

WO2014095924 discloses the pharmaceutical combination product of umeclidinium and fluticasone propionate and salmeterol xinafoate.

WO2014140648 discloses a composition of (9, 10-Dimethoxy-2-(2,4,6-trimethylphenylimino)-3-(-carbamoyl-2-aminoethyl)-3,4,6,7-tetrahydro-2H-pyrimido[6, 1-a]isoquinolin-4-one) and a muscarinic receptor antagonist such as tropine, hyoscine, glycopyrrolate, ipratropium, tiotropium, oxitropium, pirenzepine, telenzepine, aclidinium or umeclidinium.

WO2015015446 discloses umeclidinium for use in the topical treatment or prophylaxis of excessive sweating.

WO2009036243 discloses a pharmaceutical product of darotropium, salmeterol xinafoate and fluticasone propionate.

Combination therapy is always preferable in chronic conditions and would also take care of the severity and probable aggravation of these conditions. However, selection of a specific β₂ agonist and an anticholinergic agent always plays a very important role in formulation of fixed dose combinations to control respiratory disorders.

Further, selecting a combination of a β₂ agonist and an anticholinergic agent with a sustained duration of action has been found by the inventors to be critical in a treatment method where a β₂ agonist is required to be administered once daily and an anticholinergic agent is required to be administered twice daily or vice versa would usually provide a once or twice a day treatment.

Thus, the inventors have found that there is still a need to develop or formulate suitable combinations comprising a β₂ agonist and an anticholinergic agent that alleviate asthma and COPD, and that there still exists a need to formulate pharmaceutical compositions comprising a β₂ agonist and an anticholinergic agent which exhibits reduced side effects for the treatment of these respiratory disorders.

### OBJECT OF THE INVENTION

An object of the present invention is to provide a pharmaceutical composition comprising one or more bronchodilators for administration in the prevention or treatment of respiratory, inflammatory or obstructive airway disease.

Another object of the present invention is to provide a pharmaceutical composition comprising one or more bronchodilators for once or twice daily administration for the prevention or treatment of respiratory, inflammatory or obstructive airway disease.

Yet another object of the present invention is to provide a process for preparing the pharmaceutical composition comprising one or more bronchodilators for administration in the prevention or treatment of respiratory, inflammatory or obstructive airway disease.

A further object of the present invention is to provide a method for prophylaxis or treatment of asthma, COPD or a related respiratory or nasal disorder which comprises administering a pharmaceutical composition comprising one or more bronchodilators.

Another object of the present invention is to provide a pharmaceutical composition comprising one or more bronchodilators for use in the prophylaxis or treatment of asthma, COPD or a related respiratory or nasal disorder.

### SUMMARY OF THE INVENTION

According to an aspect of the present invention, there is provided a pharmaceutical composition comprising one or more bronchodilators. Preferably, the one or more bronchodilators comprise one or more anticholinergic agents and one or more β₂-agonists. Preferably, the one or more anticholinergic agents comprise umeclidinium or darotropium. Preferably, the one or more β₂-agonists comprise abediterol. Preferably, the pharmaceutical composition of the invention is suitable for once or twice daily administration to a patient in need thereof.

According to another aspect of the present invention, there is provided a pharmaceutical composition of the invention comprising one or more bronchodilators for use in the treatment of respiratory, inflammatory or obstructive airway disease or a related respiratory or nasal disorder.

According to another aspect of the invention, there is provided the use of a pharmaceutical composition of the invention in the manufacture of a medicament for the treatment of respiratory, inflammatory or obstructive airway disease, or a related respiratory or nasal disorder.

According to another aspect of the invention, there is provided a method of treating respiratory, inflammatory or obstructive airway disease, or a related respiratory or nasal disorder, wherein the method comprises administering a pharmaceutical composition of the invention to a patient in need thereof.

According to yet another aspect of the present invention, there is provided a process for preparing the pharmaceutical composition comprising of the invention, wherein the process comprises mixing one or more bronchodilators with one or more pharmaceutically acceptable excipients so as to form the pharmaceutical composition.

According to a further aspect of the present invention there is provided a pharmaceutical composition comprising of the invention for use in the prophylaxis or treatment of asthma, COPD or a related respiratory or nasal disorder.

In the methods, uses, and compositions for use according to the present invention, the disease is preferably chronic obstructive pulmonary disease (COPD) or asthma. Preferably, the uses or methods of the invention comprise once or twice daily administration of the pharmaceutical composition of the invention to a patient in need thereof.

### DETAILED DESCRIPTION OF THE INVENTION

The selection of a specific β₂-agonist and an anticholinergic agent always plays a very important role in the formulation of fixed dose combinations.

However, the combinations of such LABA's and LAMA's that are currently being developed, such as glycopyrronium and indacaterol, aclidinium and formoterol, aclidinium and glycopyrronium, tiotropium and olodaterol as well as umeclidinium and vilanterol which exhibit significant bronchodilation effect.

However, there is a constant need to develop new drugs for COPD because the global prevalence of COPD is rising and the disease is associated with significant morbidity and mortality and also the current treatment options are comparatively limited. The present invention thus, provides pharmaceutical compositions for inhalation comprising umeclidinium or darotropium as an anticholinergic agent and abediterol β₂-agonist.

Preferably, the present invention provides pharmaceutical compositions for inhalation comprising umeclidinium or darotropium as an anticholinergic agent and abediterol β₂-agonist, wherein the pharmaceutical compositions provide a duration of action for a period of 24 hours.

The inventors of the present invention have found that the above mentioned pharmaceutical compositions are effective for treating inflammatory and/or obstructive diseases of the respiratory tract, particularly asthma or chronic obstructive pulmonary disease (COPD).

Furthermore, the pharmaceutical compositions of the present invention advantageously provide a rapid onset of action, longer duration of action and improved control of obstructive or inflammatory airway diseases, or reduction in the exacerbations of the diseases.

Also, the pharmaceutical compositions of the present invention advantageously reduce the risk of undesirable side effects as compared to the repeated exposure of the steroid alone involved in the treatment of inflammatory or obstructive airways diseases.

Another advantage of the pharmaceutical compositions of the present invention is that the invention facilitates the treatment of an obstructive and inflammatory airway disease with use of a fixed dose combination in a single medicament.

Further the pharmaceutical compositions of the present invention provide for the administration of combination therapies by use of a single inhaler for patients who currently have to make use of multiple inhalers. By way of example, patients may administer pharmaceutical compositions of the present invention from a single inhaler instead of administering from two different inhalers, one for anticholinergic and one for a long acting beta₂-agonist. This is particularly important in case of elderly patients who may get confused between the inhalers and who also suffer from several other medical conditions such as heart disease and arthritis, and are receiving multiple other medications.

Preferably, the pharmaceutical compositions of the present invention are formulated for once or twice daily administration, wherein the pharmaceutical compositions provide a duration of action for a period of 24 hours.

Umeclidinium is chemically known as 6 Diphenyl-[1-(2-phenylmethoxyethyl)-1-azoniabicyclo [2.2.2] octan-4-yl] methanol bromide. A particularly preferred pharmaceutically acceptable salt of umeclidinium for use in compositions of the invention is umeclidinium bromide. According to the present invention, umeclidinium may preferably be present in an amount of from about 10 mcg to about 150 mcg.

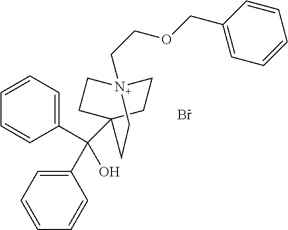 [SMILES: OC(c1ccccc1)(c1ccccc1)C12CC[N+](CCOCc3ccccc3)(CC1)CC2 ; Br]

Further, umeclidinium has a longer duration of action as compared to tiotropium bromide.

Also, it has been reported that umeclidinium exhibits bronchodilator effect which is equivalent when compared to tiotropium.

Darotropium is chemically known as 3-[(1R, 5S)-8,8-dimethyl-8-azoniabicyclo[3.2.1]octan-3-yl]-2,2-diphenylpropanenitrile; bromide. A particularly preferred pharmaceutically acceptable salt of darotropium for use in compositions of the invention is darotropium bromide. According to the present invention, darotropium may preferably be present in an amount of from about 10 mcg to about 100 mcg.

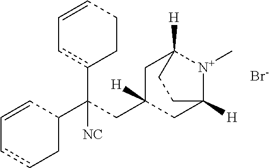

In addition to umeclidinium or darotropium, the pharmaceutical compositions of the present invention further comprise a β₂-agonist, preferably abediterol.

Abediterol is β₂-agonist and is chemically described as (R)-5-(2-((6-(2,2-difluoro-2-phenylethoxy)hexyl)amino)-1-hydroxyethyl)-8-hydroxyquinolin-2(1H)-one.

According to the present invention, abediterol may preferably be present in an amount of from about 0.1 mcg to about 10 mcg.

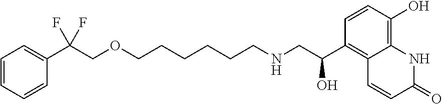 [SMILES: O=c1ccc2c([C@@H](O)CNCCCCCCOCC(F)(F)c3ccccc3)ccc(O)c2[nH]1]

In addition to umeclidinium or darotropium and abediterol, the pharmaceutical compositions of the present invention may also comprise an inhaled corticosteroid or a phosphodiesterase type 4 inhibitor, or combinations thereof.

Inhaled corticosteroids, for use in the present invention include, but are not limited to, fluticasone propionate, fluticasone valerate, fluticasone furoate, mometasone, ciclesonide, budesonide, R(+) budesonide, betamethasone, beclomethasone and the like, and combinations thereof.

Accordingly, the pharmaceutical composition of the invention can comprise umeclidinium, abediterol and ciclesonide; umeclidinium, abediterol and mometasone; umeclidinium, abediterol and fluticasone propionate; umeclidinium, abediterol and beclomethasone; umeclidinium, abediterol and budesonide; umeclidinium, abediterol and fluticasone furoate; darotropium, abediterol and beclomethasone; darotropium, abediterol and budesonide; or darotropium, abediterol and fluticasone propionate.

Phosphodiesterase type 4 inhibitors, that can be used in compositions of the invention, comprise, but are not limited to, roflumilast, cilomilast, ibudilast, tetomilast, theophylline and the like, or combinations thereof.

Accordingly, the pharmaceutical composition of the invention can comprise umeclidinium or darotropium, abediterol and roflumilast; umeclidinium or darotropium, abediterol and tetomilast; or, umeclidinium or darotropium, abediterol and theophylline.

As used herein the terms “umeclidinium”, “darotropium” and “abediterol” are used in broad sense to include not only “umeclidinium”, “darotropium” and “abediterol” per se but also their pharmaceutically acceptable derivatives. Suitable pharmaceutically acceptable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof, and combinations thereof.

The pharmaceutically acceptable salts of umeclidinium, include but are not limited to, chloride, bromide, iodide, hydroxide, sulfate, nitrate, phosphate, acetate, trifluoroacetate, fumarate, citrate, tartrate, oxalate, succinate, mandelate, methanesulfonate or p-toluenesulfonate. Preferably, umeclidinium may be in the form of umeclidinium bromide.

The pharmaceutically acceptable salts of darotropium include but are not limited to, chloride, bromide, iodide, sulfate, benzene sulfonate or toluene sulfonate. Preferably, darotropium may be in the form of darotropium bromide.

In addition to active pharmaceutical ingredients, the pharmaceutical compositions of the present invention typically comprise one or more pharmaceutically acceptable excipients. The active ingredients may be used as separate formulations or as a single combined formulation. When combined in the same formulation, it will be appreciated that the active ingredients must be stable and compatible with each other and the other components of the formulation.

The pharmaceutical compositions of the present invention are formulated for inhalation and may therefore be administered by any suitable methods used for delivery of the drugs to the respiratory tract. For example, the composition of the present invention may be in the form of an aerosol composition, a nasal spray, nasal drops or an insufflation powder. Such aerosol compositions may be administered by any conventional means, for example using a metered dose inhaler (MDI), dry powder inhaler (DPI) or nebulizer.

The various dosage forms according to the present invention may comprise carriers/excipients suitable for formulating the same.

The pharmaceutical compositions of the present invention can be in a form suitable for administration by an MDI, for example, in the form of an aerosol composition. Such compositions may comprise one or more pharmaceutically acceptable excipients, for example, those selected from the group of propellants such as HFC/HFA propellants, co-solvents, bulking agents, non-volatile components, buffers/pH adjusting agents, surface active agents, preservatives, complexing agents, or combinations thereof.

Suitable propellants are those which, when mixed with the cosolvent(s), form a homogeneous propellant system in which a therapeutically effective amount of the medicament can be dissolved. The HFC/HFA propellant must be toxicologically safe and must have a vapor pressure which is suitable to enable the medicament to be administered via a pressurized MDI.

According to the present invention, the HFC/HFA propellants may comprise, one or more of 1,1,1,2-tetrafluoroethane (HFA-134(a)) and 1,1,1,2,3,3,3,-heptafluoropropane (HFA-227), HFC-32 (difluoromethane), HFC-143(a) (1,1,1-trifluoroethane), HFC-134 (1,1,2,2-tetrafluoroethane), and HFC-152a (1,1-difluoroethane) or combinations thereof and such other propellants which may be known to the person having a skill in the art.

In the context of the present invention, the term “co-solvent” means any solvent which is miscible in the formulation in the amount desired and which, when added provides a formulation in which the medicament can be dissolved. The function of the co-solvent is to increase the solubility of the medicament and the excipients in the formulation.

According to the present invention, the co-solvent may comprise one or more of: C2-C6 aliphatic alcohols, such as, but not limited to, ethyl alcohol and isopropyl alcohol; glycols such as but not limited to propylene glycol, polyethylene glycols, polypropylene glycols, glycol ethers; block copolymers of oxyethylene and oxypropylene; and other substances, such as, but not limited to, glycerol, polyoxyethylene alcohols, and polyoxyethylene fatty acid esters; hydrocarbons such as, but not limited, to n-propane, n-butane, isobutane, n-pentane, iso-pentane, neo-pentane, and n-hexane; and ethers such as but not limited to diethyl ether; and combinations thereof.

Suitable surfactants which may be employed in an aerosol composition of the present invention include those which may serve to stabilize the solution formulation and improve the performance of valve systems of the metered dose inhaler. Preferred surfactants include one or more ionic and/or non-ionic surfactants. Examples of suitable surfactants include, but are not limited to, oleic acid, sorbitan trioleate, lecithin, isopropylmyristate, tyloxapol, polyvinylpyrrolidone, polysorbates such as polysorbate 80, vitamin E-TPGS, and macrogol hydroxystearates such as macrogol-15-hydroxystearate, and combinations thereof.

In the context of the present invention, the term “non-volatile component” refers to the suspended or dissolved constituents of the pharmaceutical composition that would remain after evaporation of the solvent(s) present.

According to the present invention, the non-volatile component may comprise one or more of monosaccharides such as, but not limited to, glucose, arabinose; disaccharides such as lactose, maltose; oligosaccharides and polysaccharides such as, but not limited to, dextrans; polyalcohol such as, but not limited to, glycerol, sorbitol, mannitol, xylitol; salts such as, but not limited to, potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate and calcium chloride and combinations thereof.

Suitable bulking agents may be employed in the pharmaceutical compositions of the invention, in particular aerosol compositions that are intended for administration using an MDI. The bulking agent may comprise one or more of saccharides, including monosaccharides, disaccharides, polysaccharides and sugar alcohols such as arabinose, glucose, fructose, ribose, mannose, sucrose, terhalose, lactose, maltose, starches, dextran or mannitol; and combinations thereof.

Suitable buffers or pH adjusting agents may be employed in the pharmaceutical compositions of the invention, in particular aerosol compositions that are intended for administration using an MDI. The buffer or the pH adjusting agent may comprise one or more of organic or inorganic acids such as, but not limited to, citric acid, ascorbic acid, hydrochloric acid, sulfuric acid, nitric acid, or phosphoric acid and combinations thereof.

Suitable preservatives may be employed in the pharmaceutical compositions of the invention, in particular aerosol compositions that are intended for administration using an MDI, to protect the formulation from contamination with pathogenic bacteria. The preservative may comprise one or more of benzalkonium chloride, benzoic acid, benzoates such as sodium benzoate and such other preservatives which may be known to the person having a skill in the art and combinations thereof.

Suitable complexing agents may be employed in the pharmaceutical compositions of the invention, in particular aerosol compositions that are intended for administration using an MDI, capable of forming complex bonds. The complexing agent may comprise one or more of, but not limited to, sodium EDTA or disodium EDTA and combinations thereof.

The pharmaceutical compositions of the present invention can be in the form suitable for administration by a dry powder inhaler (DPI).

The pharmaceutically acceptable excipients present in the compositions of the invention when suitable for dry powder inhalation comprise suitable carriers which include, but are not limited to, sugars such as glucose, saccharose, lactose and fructose, starches or starch derivatives, oligosaccharides such as dextrins, cyclodextrins and their derivatives, polyvinylpyrrolidone, alginic acid, tylose, silicic acid, cellulose, cellulose derivatives (for example cellulose ether), sugar alcohols such as mannitol or sorbitol, calcium carbonate, calcium phosphate, etc. lactose, lactitol, dextrates, dextrose, maltodextrin, saccharides including monosaccharides, disaccharides, polysaccharides; sugar alcohols such as arabinose, ribose, mannose, sucrose, trehalose, maltose, dextran, magnesium stearate, cellobiose octaacetate; and combinations thereof.

The pharmaceutical compositions of the present invention can also be in a form suitable for administration by nebulization.

Nebulization therapy is easy to use and does not require co-ordination or much effort. It also works much more rapidly than medicines taken by mouth. Such compositions may comprise suitable excipients such as one or more of, but not limited to, tonicity agents, pH regulators, chelating agents in a suitable vehicle, or combinations thereof.

Examples of suitable isotonicity adjusting agents include sodium chloride, potassium chloride, zinc chloride, calcium chloride, mannitol, glycerol, dextrose and mixtures thereof.

The pH of pharmaceutical compositions of the invention may be adjusted by the addition of one or more pH regulators such as pharmacologically acceptable acids. Pharmacologically acceptable inorganic acids or organic acids may be used for this purpose. Examples of preferred inorganic acids include one or more acids selected from the group consisting of hydrochloric acid, hydrobromic acid, nitric acid, sulphuric acid and phosphoric acid and combinations thereof. Examples of particularly suitable organic acids include one or more acids selected from the group consisting of ascorbic acid, citric acid, malic acid, tartaric acid, maleic acid, succinic acid, fumaric acid, acetic acid, formic acid and propionic acid and combinations thereof.

Examples of suitable chelating agents for use in a pharmaceutical compositions of the invention include editic acid (EDTA) or a salt thereof, e.g. sodium EDTA or disodium EDTA dihydrate (sodium edetate), and mixtures of such compounds.

Dosage forms such as nasal sprays and nasal drops of compositions of the invention may comprise isotonicity-adjusting agents, pH regulators, chelating agents, thickening agents and combinations thereof, such as those used in nebulization therapy discussed above.

Examples of suitable thickening agents for use in a pharmaceutical compositions of the invention include cellulose derivatives (for example cellulose ether) in which the cellulose-hydroxy groups are partially etherized with lower unsaturated aliphatic alcohols and/or lower unsaturated aliphatic oxyalcohols (for example methyl cellulose, carboxymethyl cellulose, hydroxypropylmethylcellulose), gelatin, polyvinylpyrrolidone, tragacanth, ethoxose (water soluble binding and thickening agents on the basis of ethyl cellulose), alginic acid, polyvinyl alcohol, polyacrylic acid, pectin and equivalent agents, or combinations thereof. Should these substances contain acid groups, the corresponding physiologically acceptable salts may also be used.

In addition to the aforementioned excipients, one or more anti-microbial preservative agents may also be added to the pharmaceutical compositions of the invention, in particular for multi-dose packages.

The composition according to the present invention may be included in one or more suitable containers provided with means enabling the application of the contained formulation to the respiratory tract.

Where the pharmaceutical compositions of the invention are in the form of a powder for inhalation and are intended to be administered by a DPI, it may be encapsulated in capsules of gelatin or HPMC, or in blisters. In an alternative embodiment, the dry powder may be contained as a reservoir either in a single dose or multi-dose dry powder inhalation device. In a further alternative embodiment, the powder for inhalation may be suspended in a suitable liquid vehicle and packed in an aerosol container along with suitable propellants or mixtures thereof. In still a further alternative embodiment, the powder for inhalation may be dispersed in a suitable gas stream to form an aerosol composition.

Where the pharmaceutical compositions of the invention are in the form of an aerosol composition for administration using an MDI, it may be packed in plain aluminium cans or SS (stainless steel) cans or any such cans suitable for MDI delivery. Some aerosol drugs tend to adhere to the inner surfaces, i.e., walls of the cans and valves, of the MDI. This can lead to the patient getting significantly less than the prescribed amount of the active agent upon each activation of the MDI. Such cans may be suitably treated to avoid any adherence of the active on the walls thereof using techniques known in the art, for example coating the inner surface of the container with a suitable polymer can reduce this adhesion problem. Suitable coatings include fluorocarbon copolymers such as FEP-PES (fluorinated ethylene propylene and polyethersulphone) and PFA-PES (perfluoroalkoxyalkane and polyethersulphone), epoxy and ethylene. Alternatively, the inner surfaces of the cans may be anodized, plasma treated or plasma coated.

Where the pharmaceutical compositions of the invention are in the form of nasal sprays and nasal drops for administration into the nasal passages it may be done by means of a dropper (or pipette) that includes a glass, plastic or metal dispensing tube. Fine droplets and sprays can be provided by an intranasal pump dispenser or squeeze bottle as well known in the art.

The pharmaceutical compositions of the present invention may further comprise, in addition to those pharmaceutically active ingredients detailed above, one or more active(s) selected from the group comprising of, antihistamines, antiallergics, leukotriene antagonists, or their pharmaceutically acceptable salts, solvates, tautomers, derivatives, enantiomers, isomers, hydrates, prodrugs or polymorphs thereof, or combinations thereof.

The pharmaceutical compositions of the present invention comprise umeclidinium or darotropium and abediterol. These active ingredients are formulated for simultaneous, separate or sequential administration. When the active ingredients are administered sequentially, either umeclidinium or darotropium, or abediterol, may be administered first. When administration is simultaneous, the active ingredients may be administered either in the same or different pharmaceutical compositions. Adjunctive therapy, i.e. where one active ingredient is used as the primary treatment and the other active ingredient(s) is/are used to assist that primary treatment is also an embodiment of the present invention.

According to a further embodiment of the invention, there is provided a product comprising umeclidinium or darotropium and abediterol as a combined preparation for simultaneous, separate or sequential use for treatment and/or prevention of respiratory, inflammatory or obstructive airway disease.

Compositions for use according to the present invention may be presented in a pack or dispenser device which may contain one or more unit dosage forms containing the active ingredients. These may for example, comprise metal or plastic foil, such as a blister pack. Where compositions are intended for administration as two separate compositions these may be presented in the form of a twin pack.

Pharmaceutical compositions may also be prescribed in “patient packs” containing the whole course of treatment in a single package. The inclusion of a package insert has been shown to improve patient compliance with the prescribing physician's instructions. According to a further embodiment of the present invention, there is provided a patient pack comprising at least one active ingredient of the combination according to the invention and an information insert containing directions to use the combination of the invention. In one embodiment, the present invention provides a fixed dose combination.

The pharmaceutical compositions of the present invention may be conveniently presented in unit dosage form and may be prepared by any of the methods well known in the art. Suitable methods include the step of bringing into association the active ingredients with a carrier which constitutes one or more pharmaceutically acceptable excipients. In general, compositions may be prepared by uniformly and intimately bringing into association the active ingredients with one or more liquid carriers or finely divided solid carriers, or both. It will be appreciated that when the active ingredients are administered independently, each may be administered by a different means.

The present invention also provides a process to manufacture the compositions according to the present invention.

The present invention provides a process of preparing pharmaceutical compositions for administration by a metered dose inhaler, which process comprises admixing a pharmaceutically acceptable carrier or excipient with one or more active pharmaceutical ingredients of the invention and a propellant, and thereafter transferring the composition to a suitable container, preferably a pre-crimped can.

The invention provides a process of preparing a pharmaceutical compositions for administration by dry powder inhalation, which process comprises admixing of a pharmaceutically acceptable carrier or excipient with one or more active pharmaceutical ingredients of the invention and providing the composition as a dry powder.

The invention provides a process of preparing pharmaceutical compositions for administration by nebulisation, which process comprises dissolving the drugs, optionally chelating agents, osmotic/isotonicity adjusting agents and any other suitable ingredients in the vehicle and adjusting the pH using a suitable pH adjusting agent.

The invention also provides a method for the prevention and/or treatment of a respiratory, inflammatory or obstructive airway disease, in particular chronic obstructive pulmonary disease, in a mammal, such as a human, which method comprises administration of a therapeutically effective amount of pharmaceutical compositions according to the present invention.

The present invention also provides pharmaceutical compositions according to the present invention for use in preventing and/or treating disorders or conditions that respond to, or are prevented, ameliorated or eliminated by, the administration one or more bronchodilators and an inhaled corticosteroid (ICS), such as a respiratory, inflammatory or obstructive airway disease, in particular chronic obstructive pulmonary disease.

The following examples are for the purpose of illustration of the invention only and are not intended in any way to limit the scope of the present invention.

### Example 1

| Sr. No. | Ingredients | Qty/Spray |
|---|---|---|
| 1. | Umeclidinium Bromide | 62.5 mcg |
| 2. | Abediterol | 2.5 mcg |
| 3. | HFA134A OR HFA227 | q.s |

Process:

1) Umeclidinium bromide and abediterol were homogenized with part quantity of HFA.
2) The suspension obtained in step (1) was transferred to the mixing vessel where remaining quantity of HFA was added.
3) The resulting suspension was mixed, recirculated and filled in into pre-crimped aluminum cans.

### Example 2

| Sr. No. | Ingredients | Qty/Spray |
|---|---|---|
| 1. | Umeclidinium Bromide | 62.5 mcg |
| 2. | Abediterol | 2.5 mcg |
| 4. | Lactose | 100% of the drug |
| 5. | HFA134A OR HFA227 | q.s. |

Process:

1) Umeclidinium bromide and abediterol were homogenized with lactose and part quantity of HFA.
2) The suspension obtained in step (1) was transferred to the mixing vessel where remaining quantity of HFA was added.
3) The resulting suspension was mixed, recirculated and filled in into pre-crimped aluminum cans.

### Example 3

| Sr. No. | Ingredients | Qty/Spray |
|---|---|---|
| 1. | Umeclidinium Bromide | 62.5 mcg |
| 2. | Abediterol | 2.5 mcg |
| 3. | PEG 400/1000 | 0.3% of total formulation |
| 4. | PVP K 25 | 0.001% of total formulation |
| 5. | HFA134A OR HFA227 | q.s. |

Process:

1) PVP and PEG were dissolved in part quantity of HFA.
2) Umeclidinium bromide and abediterol were homogenized with part quantity of HFA.
2) The suspension obtained in step (1) and (2) was transferred to the mixing vessel where remaining quantity of HFA was added.
3) The resulting suspension was mixed, recirculated and filled in into pre-crimped aluminum cans.

### Example 4

| Sr. No. | Ingredients | Qty/Spray |
|---|---|---|
| 1. | Umeclidinium Bromide | 62.5 mcg |
| 2. | Abediterol | 2.5 mcg |
| 3. | Ethanol | 15%-20% of total |
| | | formulation |
| 4. | Glycerol | 1% of total formulation |
| 5. | HCL (0.08N) | pH 2.5-3.5 |
| 6. | HFA134A | q.s. |

Process:

1) Glycerol was dissolved in ethanol and required quantity of HCl was added.
2) Umeclidinium bromide and abediterol were dissolved in the solution obtained in step 1.
3) The resulting solution was transferred to the mixing vessel where HFA was added.
4) The resulting suspension was mixed, recirculated and filled in into pre-crimped aluminum cans.

### Example 5

| Sr. No. | Ingredients | Qty/Spray |
|---|---|---|
| 1. | Umeclidinium Bromide | 62.5 mcg |
| 2. | Abediterol | 2.5 mcg |
| 3. | Ethanol | 1%-2% of total formulation |
| 4. | Lecithin | 0.02 of the API |
| 6. | HFA134a or HFA227 | q.s. |

Process:

1) Lecithin was dissolved in ethanol.
2) Umeclidinium bromide and abediterol were homogenized with part quantity of HFA and transferred to the mixing vessel.
3) The solution of lecithin and ethanol homogenized with part quantity of HFA.
4) The suspension obtained instep (3) was transferred to the main mixing vessel where the remaining quantity of HFA was added.
5) The resulting suspension was mixed, recirculated and filled in into pre-crimped aluminum cans.

### Example 6

| Sr. No. | Ingredients | Qty/Spray |
|---|---|---|
| 1. | Umeclidinium Bromide | 62.5 mcg |
| 2. | Abediterol | 2.5 mcg |
| 4. | Ethanol | 1%-2% of total formulation |
| 5. | Oleic acid | 0.02%-5% of the API |
| 6. | HFA134a or HFA227 | q.s. |

Process:

1) Oleic acid was dissolved in ethanol.
2) Umeclidinium bromide and abediterol were homogenized with part quantity of HFA and transferred to the mixing vessel.
3) The solution of oleic acid and ethanol homogenized with part quantity of HFA.
4) The suspension obtained in step (3) was transferred to the main mixing vessel where the remaining quantity of HFA was added.
5) The resulting suspension was mixed, recirculated and filled in into pre-crimped aluminum cans.

### Example 7

| Sr. No. | Ingredients | Qty/Spray |
|---|---|---|
| 1. | Umeclidinium Bromide | 62.5 mcg |
| 2. | Abediterol | 2.5 mcg |
| 4. | Ethanol | 15%-20% of total formulation |
| 5. | Glycerol | 1% of total formulation |
| 6. | HCL (0.08N) | pH 2.5-3.5 |
| 7. | HFA134A | q.s. |

Process:

1) Glycerol was dissolved in ethanol and required quantity of HCl was added.
2) Umeclidinium Bromide and abediterol were dissolved in the solution obtained in step 1.
3) The resulting solution was transferred to the mixing vessel where HFA was added.
4) The resulting suspension was mixed, recirculated and filled in into pre-crimped aluminum cans.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1. A pharmaceutical composition comprising one or more bronchodilators, or one or more pharmaceutically acceptable derivatives thereof, and one or more pharmaceutically acceptable excipients.

2. (canceled)

3. The pharmaceutical composition according to claim 1, wherein the one or more bronchodilators comprise one or more β₂-agonists and one or more anticholinergic agents.

4. The pharmaceutical composition according to claim 3, wherein the one or more β₂-agonists comprise abediterol.

5. The pharmaceutical composition according to claim 3, wherein the one or more anticholinergic agents comprise umeclidinium or darotropium.

6. The pharmaceutical composition according to claim 3, wherein the one or more anticholinergic agents comprise umeclidinium or darotropium and the one or more β₂-agonists comprise abediterol.

7. The pharmaceutical composition according to claim 5, wherein the umeclidinium is present in the form of umeclidinium bromide.

8. The pharmaceutical composition according to claim 5, wherein the darotropium is present in the form of darotropium bromide.

9. The pharmaceutical composition according to claim 5, wherein the umeclidinium is present in the composition in an amount of from 10 mcg to 150 mcg.

10. The pharmaceutical composition according to claim 5, wherein the darotropium is present in the composition in an amount of from 10 mcg to 100 mcg.

11. The pharmaceutical composition according to claim 4, wherein the abediterol is present in an amount of from 0.1 mcg to 10 mcg.

12. (canceled)

13. The pharmaceutical composition according to claim 1, wherein the composition further comprises one or more inhaled corticosteroids, one or more phosphodiesterase type 4 inhibitors, one or more antihistamines, one or more antiallergics, one or more leukotriene antagonists, any combination thereof, or any pharmaceutically acceptable derivative thereof.

14. The pharmaceutical composition according to claim 13, wherein the inhaled corticosteroid comprises fluticasone propionate, fluticasone valerate, fluticasone furoate, mometasone, ciclesonide, budesonide, R(+) budesonide, betamethasone or beclomethasone, or any combination thereof.

15. The pharmaceutical composition according to claim 13, wherein the phosphodiesterase type 4 inhibitor comprises roflumilast, cilomilast, ibudilast, tetomilast or theophylline, or any combination thereof.

16. The pharmaceutical composition according to claim 1, wherein the composition is in the form of a metered dose inhaler (MDI), dry powder inhaler (DPI), nebulizer, nasal spray, nasal drops, or an insufflation powder.

17. The pharmaceutical composition according to claim 1, formulated for use in a metered dose inhaler comprising a propellant.

18. The pharmaceutical composition according to claim 16, further comprising one or more excipients selected from a cosolvent, an antioxidant, a surfactant, a bulking agent, a lubricant, or any combination thereof.

19. The pharmaceutical composition according to claim 1, formulated for use as a dry powder inhaler, preferably comprising at least one finely divided pharmaceutically acceptable carrier suitable for use in dry powder inhaler.

20. The pharmaceutical composition according to claim 1, formulated for use as an inhalation solution/suspension.

21. The pharmaceutical composition according to claim 20, further comprising one or more excipients selected from a wetting agent, osmotic agent, a pH regulator, a buffering agent and a complexing agent provided in a pharmaceutically acceptable vehicle, or any combination thereof.

22. (canceled)

23. The process for preparing a pharmaceutical composition according to claim 1, wherein the process comprises mixing one or more bronchodilators with one or more pharmaceutically acceptable excipients so as to form the pharmaceutical composition.

24. (canceled)

25. A process comprising utilizing the pharmaceutical composition according to claim 1, in the manufacture of a medicament for the treatment of respiratory, inflammatory or obstructive airway disease, or a related respiratory or nasal disorder.

26. A method of treating respiratory, inflammatory or obstructive airway disease, or a related respiratory or nasal disorder, wherein the method comprises administering a pharmaceutical composition according to claim 1 to a patient in need thereof.

27. The method according to claim 26, wherein the disease is COPD or asthma.

28-30. (canceled)
